(19) United States
(12) Patent Application Publication
(10) Pub. No.: US 2023/0120630 A1
(43) Pub. Date: Apr. 20, 2023

(54) VAPORIZER WITH SPACED HEATING MODULES

(71) Applicant: Iconic Ventures, Inc., Portland, OR (US)

(72) Inventor: Michael Lindars, Portland, OR (US)

(21) Appl. No.: 17/875,320

(22) Filed: Jul. 27, 2022

Related U.S. Application Data
(63) Continuation-in-part of application No. 16/945,801, filed on Jul. 31, 2020.
(60) Provisional application No. 63/225,998, filed on Jul. 27, 2021.
(60) Provisional application No. 62/835,950, filed on Apr. 18, 2019.
(60) Provisional application No. 62/843,561, filed on May 6, 2019.

(51) Int. Cl. A24F 40/46; H05B 3/06; H05B 1/02; A24F 40/10; A24F 40/42; A24F 40/57; A61M 11/04

(52) U.S. Cl. CPC A24F 40/46; H05B 3/06; H05B 1/0244; A24F 40/10; A24F 40/42; A24F 40/57; A61M 11/042; H05B 2203/005

(57) ABSTRACT

A cartridge for a vaporizer can include an elongated reservoir body and a plurality of longitudinally spaced heating modules configured to heat corresponding portions or zones of the reservoir body for vaporizing a vaporizable substance disposed therein. The plurality of heating modules can be individually controlled or controllable for optimizing user control over a vaporizer operation. The reservoir body can be disposed in or through openings in the heating modules with a gap there between and the heating modules can be configured to heat the reservoir body from a plurality of directions.

### CROSS REFERENCE TO RELATED APPLICATIONS

This application claims the benefit of US Provisional Patent Application No. 63/225,998 filed Jul. 27, 2021, the entire contents of which are hereby incorporated herein by reference. This application is a continuation in part of U.S. patent application Ser. No. 16/945,801 filed Jul. 31, 2020, which claims the benefit of U.S. Provisional Patent Application Nos. 62835950 filed Apr. 18, 2019 and 62/843,561 filed May 6, 2019, the entire contents of which are hereby incorporated by reference into this disclosure.

### STATEMENT REGARDING FEDERALLY SPONSORED RESEARCH OR DEVELOPMENT

Not applicable.

### REFERENCE TO APPENDIX

Not applicable.

### BACKGROUND

The present disclosure relates generally to vaporizers and more specifically relates to vaporizers for converting oil to a mist for inhalation by a user. A vaporizer can be used to convert oil or another substance, such as a substance that contains medication or other compounds, to a vapor or mist for inhalation by a user. However, at least some vaporizers suffer from one or more shortcomings, such as burning or carbonization of the oil, excessive wicking, leaking, or clogging, and/or imprecise control features. A need exists in the art for improved devices, systems and methods for vaporizing oil and/or other vaporizable substances.

### BRIEF SUMMARY

A cartridge for a vaporizer can include an elongated reservoir body and a plurality of longitudinally spaced heating modules configured to heat corresponding portions or zones of the reservoir body for vaporizing a vaporizable substance disposed therein. The plurality of heating modules can be individually controlled or controllable for optimizing user control over a vaporizer operation. The reservoir body can be disposed in or through openings in the heating modules with a gap there between and the heating modules can be configured to heat the reservoir body from a plurality of directions.

In at least one embodiment, a cartridge for a vaporizer can include a reservoir for holding a vaporizable substance and a plurality of heating modules. In at least one embodiment, the reservoir can include an elongated reservoir body having a longitudinal axis, a first end and a second end longitudinally opposite the first end. In at least one embodiment, the plurality of heating modules can include a first heating module and a second heating module. In at least one embodiment, the first heating module can be configured to heat a first portion of the reservoir body and the second heating module can be configured to heat a second portion of the reservoir body. In at least one embodiment, the first heating module can be disposed at a first location along the longitudinal axis of the reservoir body. In at least one embodiment, the second heating module can be disposed at a second location along the longitudinal axis of the reservoir body. In at least one embodiment, the second heating module can be longitudinally spaced from the first heating module. In at least one embodiment, the cartridge can further include a support tube between the first and second heating modules.

In at least one embodiment, the plurality of heating modules can further include one or more additional heating modules disposed at one or more respective additional locations along the longitudinal axis of the reservoir body and configured to heat one or more respective additional portions of the reservoir body. In at least one embodiment, the one or more additional heating modules can be longitudinally spaced from the first heating module and the second heating module. In at least one embodiment, the spacing between adjacent ones of the plurality of heating modules can be uniform. In at least one embodiment, the spacing between the first and second heating modules can be different from the spacing between the second heating module and at least one of the one or more additional heating modules. In at least one embodiment, at least one of the plurality of heating modules can be a radiant heat source disposed in thermal communication with the reservoir body. In at least one embodiment, the radiant heat source can be separated from the reservoir body by an air gap. In at least one embodiment, the radiant heat source can be configured to heat the reservoir body from at least two directions.

In at least one embodiment, each of the plurality of heating modules can comprise an annular heating body having an opening therethrough and wherein the reservoir body can be disposed through the openings of the annular heating bodies. In at least one embodiment, the reservoir body can be suspended within the openings of the annular heating bodies with an air gap between the exterior surface of the reservoir body and the interior surfaces of the annular heating bodies. In at least one embodiment, the reservoir body can be cylindrical and/or can be suspended from its ends.

In at least one embodiment, the cartridge can further include one or more reflectors configured to reflect heat from one or more of the plurality of heating modules. In at least one embodiment, the cartridge can further include one or more heat sinks in thermal communication with one or more of the plurality of heating modules. In at least one embodiment, the cartridge can further include one or more end caps configured to couple to at least one end of the reservoir body for holding the reservoir body in place relative to one or more of the plurality of heating modules. In at least one embodiment, the reservoir body can include a porous body configured to store a vaporizable substance in a plurality of internal pores.

In at least one embodiment, the cartridge can further include a controller operably coupled to the first and second heating modules. In at least one embodiment, the controller can be configured for selective and/or independent activation or control of one or both of the first and second heating modules. In at least one embodiment, the controller can be configured to selectively heat the reservoir body to a first temperature for a first time period; reduce an amount of heat applied to the reservoir body; maintain the reservoir body at a second temperature for a second time period; or any combination thereof.

In at least one embodiment, the controller can be configured to simultaneously operate one or both of the first and second heating modules at a first heating level, simultaneously operate one or both of the first and second heating modules at a second heating level less than the first heating level, and thereafter selectively operate one or both of the first and second heating modules at a third heating level.

In at least one embodiment, a cartridge for a vaporizer can include a reservoir for holding a vaporizable substance. In at least one embodiment, the reservoir can comprise an elongated reservoir body having a longitudinal axis, a first end and a second end longitudinally opposite the first end. In at least one embodiment, a cartridge for a vaporizer can include a first heating module disposed at a first location along the longitudinal axis of the reservoir body. In at least one embodiment, a cartridge for a vaporizer can include a second heating module disposed at a second location along the longitudinal axis of the reservoir body. In at least one embodiment, a cartridge for a vaporizer can include a first support tube disposed at a third location along the longitudinal axis of the reservoir body between the first and second heating modules. In at least one embodiment, a cartridge for a vaporizer can include a third heating module disposed at a fourth location along the longitudinal axis of the reservoir body. In at least one embodiment, a cartridge for a vaporizer can include a second support tube disposed at a fifth location along the longitudinal axis of the reservoir body between the second and third heating modules. In at least one embodiment, a cartridge for a vaporizer can include a controller operably coupled to the heating modules. In at least one embodiment, the controller can be configured for selective and/or independent activation of the heating modules. In at least one embodiment, the controller can be configured to selectively heat different portions of the reservoir.

### BRIEF DESCRIPTION OF THE SEVERAL VIEWS OF THE DRAWINGS

FIG. 1 is a perspective view of one of many embodiments of a vaporizer according to the disclosure.

FIGS. 2-5 and 7-9 are perspective views of some of many embodiments of a vaporization assembly according to the disclosure.

FIG. 6 is a perspective view of one of many embodiments of a support tube according to the disclosure.

FIG. 10 is a block diagram of one of many embodiments of select components of a vaporizer according to the disclosure.

### DESCRIPTION

The Figures described above and the written description of specific structures and functions below are not presented to limit the scope of what Applicant has invented or the scope of the appended claims. Rather, the Figures and written description are provided to teach any person skilled in the art to make and use the invention(s) for which patent protection is sought. Those skilled in the art will appreciate that not all features of a commercial embodiment of the disclosure are described or shown for the sake of clarity and understanding. Persons of skill in this art will appreciate that the development of an actual commercial embodiment incorporating aspects of the present disclosure can require numerous implementation-specific decisions to achieve the developer's ultimate goal for the commercial embodiment(s). Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in the art having the benefits of this disclosure.

The embodiment(s) disclosed and taught herein are susceptible to numerous and various modifications and alternative forms. The use of a singular term, such as, but not limited to, “a,” is not intended as limiting of the number of items. The use of relational terms, such as, but not limited to, “top,” “bottom,” “front,” “rear,” “left,” “right,” “upper,” “lower,” “down,” “up,” “side,” “first,” “second,” “inlet,” “outlet” and the like are used in the written description for clarity in specific reference to the Figures and are not intended to limit the scope of the disclosure or the appended claims unless otherwise indicated. The terms “couple,” “coupled,” “coupling,” “coupler,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, operably, directly or indirectly with intermediate elements, one or more pieces of members together and can further include without limitation integrally forming one member with another in a unity fashion. The coupling can occur in any direction, including rotationally. The terms “include” and “such as” are illustrative and not limitative, and the word “can” means “can, but need not” unless otherwise indicated. The term “end” can, but need not, be or include a terminal end unless otherwise indicated. Notwithstanding any other language in the present disclosure, the embodiment(s) shown in the drawings are examples presented for purposes of illustration and explanation and are not the only embodiments of the subject(s) hereof.

Applicant has created devices, systems and methods for advantageously vaporizing oil or other vaporizable substances for human inhalation. Examples of such substances include, but are not limited to, cannabidiol (CBD) oil and derivatives thereof, tetrahydrocannabinol (THC) oil, liquid medication solutions and liquid nicotine solutions (aka “e-juice” or “e-liquids”), among others. In at least one embodiment, a device or system for vaporizing, such as a vaporizer or cartridge, can include a reservoir body for holding a vaporizable substance and a plurality of heaters or heating modules spaced along the reservoir body for selectively heating two or more portions or zones of the reservoir body. This can advantageously provide a user with greater control over vaporizer operations, including with regard to the volume of vapor generated during a given operation.

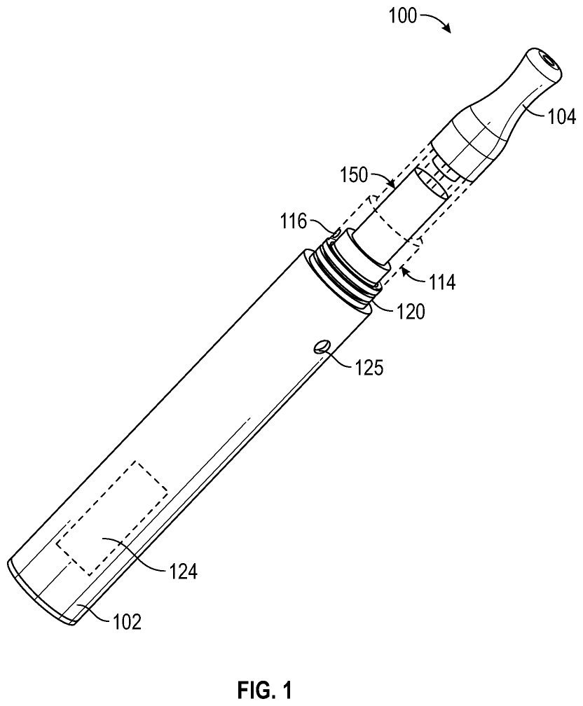

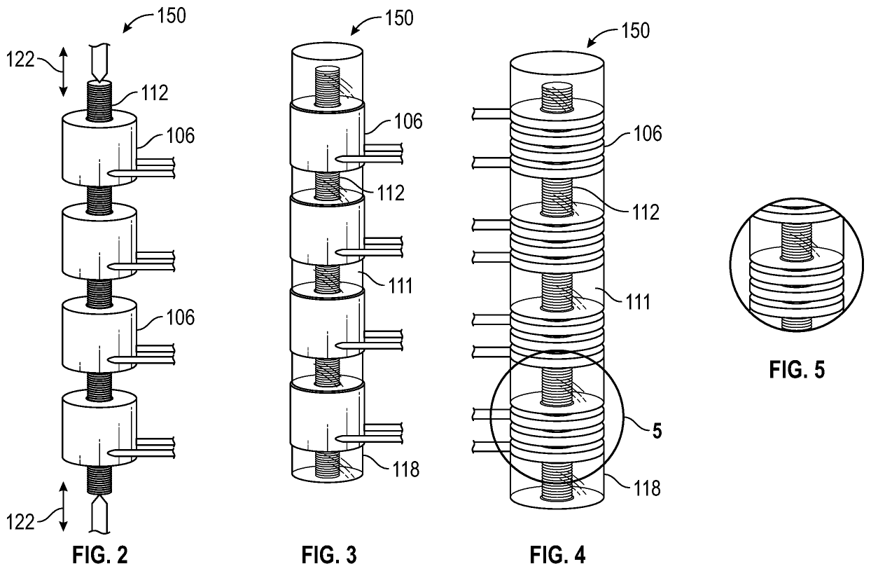

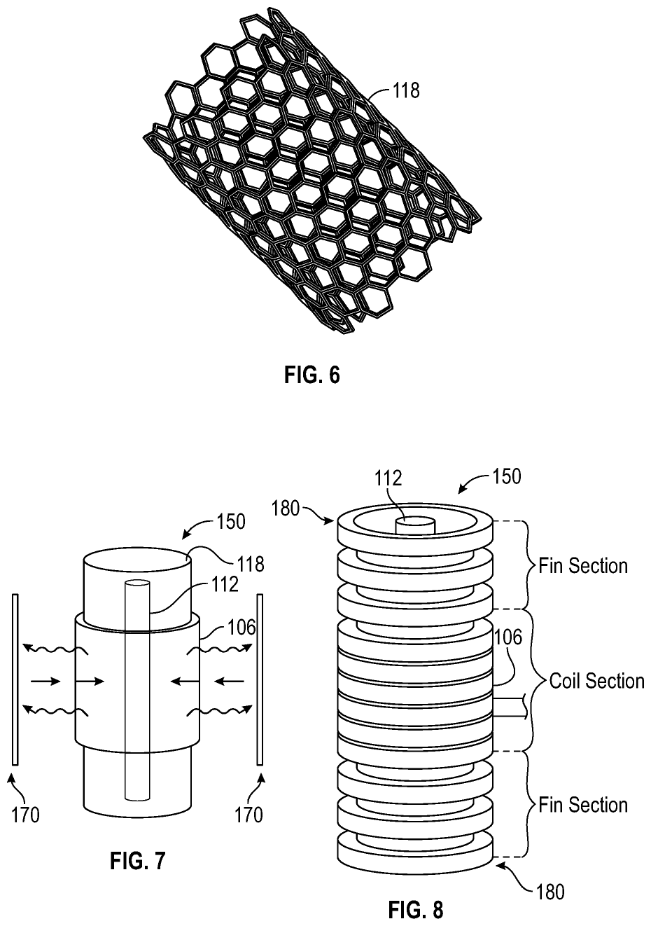

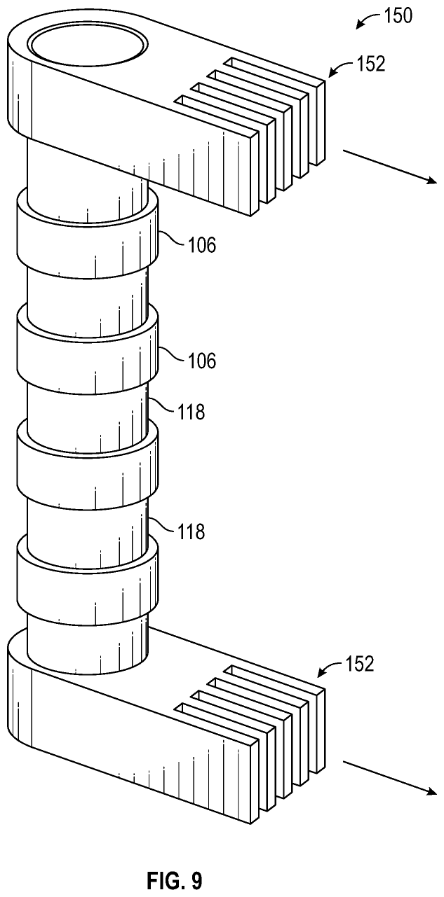

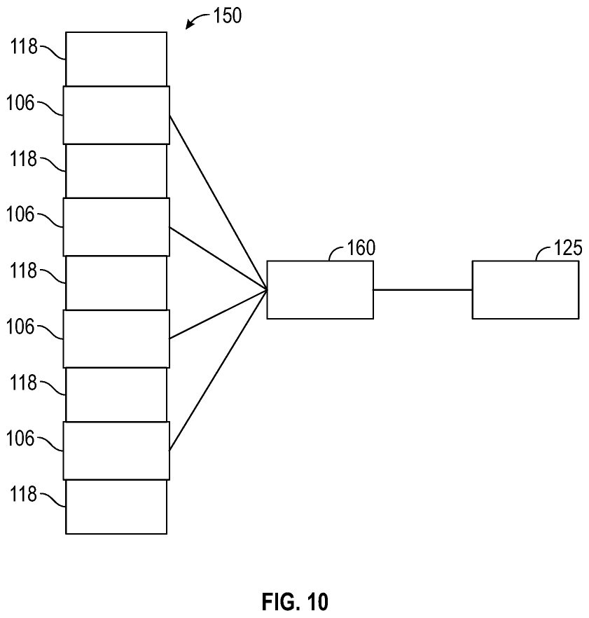

FIG. 1 is a perspective view of one of many embodiments of a vaporizer according to the disclosure. FIGS. 2-5 and 7-9 are perspective views of some of many embodiments of a vaporization assembly according to the disclosure. FIG. 6 is a perspective view of one of many embodiments of a support tube according to the disclosure. FIG. 10 is a block diagram of one of many embodiments of select components of a vaporizer according to the disclosure. FIGS. 1-10 are described in conjunction with one another.

In at least one embodiment, a vaporizer 100, such as a system for vaporizing oil, can include a body 102, such as a housing or cover, for at least partially housing or otherwise supporting one or more other components of vaporizer 100. Vaporizer 100 can include a mouthpiece 104 for routing fluid and communicating with a user's mouth or lips during use of vaporizer 100. For example, mouthpiece 104 can be configured to couple with a user's lips for directing vapor and/or air from within vaporizer 100 or a portion thereof to a user for inhalation. Vaporizer 100 can include one or more inlets 116, such as an opening or port, for allowing air to flow into or out of one or more components of vaporizer 100, such as vapor chamber 114, and one or more flow passages 122 for routing or otherwise directing fluid flow in or through vaporizer 100 or one or more portions thereof. In at least one embodiment, vaporizer 100 can include one or more power supplies 124, which can include a battery or plurality of batteries, for powering a heater or one or more other components of vaporizer 100. Vaporizer 100 can include one or more actuators 125, such as a button, switch or other control, for activating and/or deactivating the device, such as by way of controlling electrical or other communication between power supply 124 and one or more other components, such as a controller or heater. Vaporizer 100 can further include a vaporization assembly 150 comprising one or more reservoir bodies 112 and one or more heaters 106, as further described below.

In at least one embodiment, a vaporizer or a portion thereof, such as a cartridge for a vaporizer, can include a reservoir 111 for holding a vaporizable substance and a plurality of heaters or heating modules 106 for heating corresponding portions of the reservoir 111 or of one or more component(s) disposed in fluid communication with the reservoir. For instance, a reservoir 111 can include a reservoir body having a longitudinal axis, a first end and a second end longitudinally opposite the first end and the plurality of heating modules can be configured for heating corresponding portions of the reservoir body. The plurality of heating modules 106 can include two or more heating modules 106, which can include any number of heating modules 106 according to an implementation of the disclosure, such as 3, 4, 5, 6, 7, 8 . . . n heating modules. The heating modules 106 can be the same or different from one another and can be unitary or coupled to one another to form a heating module assembly or system with a plurality of heat sources spaced apart from one another according to the present disclosure.

A reservoir according to the disclosure can include one or more elongated reservoir bodies 112 configured to store a vaporizable substance and to be heated during vaporizer operations. In at least one embodiment, a reservoir body 112 according to the disclosure can be or include a porous body configured to store a vaporizable substance in a plurality of internal pores. Exemplary materials and pore sizes are further discussed elsewhere in this disclosure. A reservoir body 112 according to the disclosure can be any size and shape required or desired for an implementation of the disclosure and can preferably be an elongated rod (which can include having a solid cross-section or a tubular cross-section, in whole or in part). In at least one embodiment, a reservoir body 112 can be a cylindrical rod. For example, a reservoir body 112 can be a cylindrical rod having a length of 1.25 inches and a diameter of 0.125 inch. However, this is but one example of many; other examples include, but are not limited, lengths of from 0.5 inch to 6 inches and diameters (or other major dimensions, e.g., if not circular in cross section) from 0.0625 inch to 1 inch.

In at least one embodiment, the reservoir 111 can further include a liquid storage tank or container having an internal space in fluid communication with one or more reservoir bodies. A vaporizable substance can be stored in the tank in liquid form and can be transferred to a reservoir body (e.g., by capillary action) to refill or replenish the reservoir body with vaporizable substance as the reservoir body is emptied during vaporizer operations. In at least one embodiment, a reservoir body 112 can be at least partially tubular and vaporizable substance can be stored within the interior walls or surface of the tubular portion of the reservoir body 112. In other words, in such an embodiment, which is but one of many, a reservoir body 112 can include an internal or integral liquid storage tank, whether separately or in combination with a liquid storage tank disposed exterior of the reservoir body 112. As another example, in at least one embodiment, a reservoir body 112 can be at least partially tubular or hollow and a vaporizable substance disposed therein can be or include organic matter such as plant material stored within the interior walls or surface of the tubular portion of the reservoir body 112.

In at least one embodiment, a plurality of heating modules 106 can include a first heating module and a second heating module. The first heating module can be configured to heat a first portion of the reservoir body 112 and the second heating module can be configured to heat a second portion of the reservoir body 112. The first heating module can be disposed at a first location along the longitudinal axis of the reservoir body 112 and the second heating module can be disposed at a second location along the longitudinal axis of the reservoir body 112. The second heating module can be longitudinally spaced from the first heating module. The plurality of heating modules 106 can include one or more additional heating modules disposed at one or more respective additional locations along the longitudinal axis of the reservoir body 112 and configured to heat one or more respective additional portions of the reservoir body 112. The one or more additional heating modules 106 can be longitudinally spaced from the first heating module and the second heating module. Each heating module 106 can be configured to heat a corresponding length or zone of the reservoir or reservoir body 112, which can include solely heating a corresponding heat zone or which can include at least some overlap in adjacent heat zones due to, e.g., heat transfer through the reservoir or reservoir body.

In at least one embodiment, the plurality of heating modules 106 can be spaced apart from one another and there can be one or more cool zones disposed between adjacent hot or heating zones. It will be understood that such cool zones are not necessarily cool in terms of temperature during vaporizer operations but rather that the cool zones can be or include areas along the reservoir body 112 wherein heat is not directly applied during heating operations. The spacing between any two heating modules 106 can be any spacing according to an implementation of the disclosure and preferably can be a spacing or other orientation for minimizing or eliminating thermal loss from one heating module to another. In other words, for example, in at least one embodiment having adjacent heating modules 106, one or more of such modules can be controlled independently and it can be desirable at times for a user to operate only a single module (or another number of modules less than the total number of modules included in the embodiment) for controlling the output of vapor during device operation. In such an embodiment (among others), two or more heating modules 106 can be sufficiently spaced from one another for supporting maximum heat transfer from a module to the reservoir body 112 or vaporizable substance, which can include by way of minimizing heat transfer from an activated (or “hot”) heating module to a deactivated (or “cold”) heating module. The spacing between two or more adjacent (or other) heating modules 106 can be uniform or can differ from one zone to another. In at least one embodiment, the spacing between first and second heating modules 106 can be different from the spacing between one or both of the first and second heating modules 106 and at least one of one or more additional heating modules 106.

In at least one embodiment, one or more of a plurality of heating modules 106 can be or include a radiant heating module, such as an alumina-based module having a resistive heating element (e.g., a nichrome or other wire or coil). A heating module 106 can include a base, such as a substrate or frame, for supporting one or more heat sources and one or more heat sources for being disposed in thermal communication with the reservoir 111 or a portion thereof, such as one or more reservoir bodies 112 for being heated and supporting the creation and release of vapor during vaporizer operations. One or more radiant (or other) heat sources can be separated from the reservoir body 112 by a distance or gap, such as an air gap, which can help prevent burning or carbonization of the vaporizable substance. A heating module 106 can include one or more radiant heat sources configured to heat the reservoir body 112 from at least two directions, which can include from the top, from the bottom, from one or more sides, from inside to outside and from outside to inside, separately or in combination, in whole or in part. A heating module 106 can include one or more radiant heat sources configured to heat the reservoir body 112 from the exterior surface inwardly (or outwardly) and from 360 degrees (i.e., from all or at least substantially all directions) about an axis of the reservoir body, or some portion thereof.

In at least one embodiment, one or more of a plurality of heating modules 106 can include an annular heating body having an opening therethrough and the reservoir body 112 can be disposed at least partially in or through the opening of the annular heating body. An annular heating body according to the disclosure can be continuous or can include two or more segments disposed in a ring or ring-like manner. In at least one embodiment, each of a plurality of heating modules 106 can include an annular heating body having an opening therethrough and the reservoir body 112 can be disposed at least partially in or through the openings of the annular heating bodies. For instance, the reservoir body 112 can be suspended within the openings of the annular heating bodies with an air gap between the exterior surface of the reservoir body 112 and the interior surfaces of the annular heating bodies. In at least one embodiment, one or more reservoir bodies can be or include an elongated body, such as a shaft or rod, which can be suspended from its ends or otherwise held in place relative to the plurality of annular heating bodies. The reservoir body 112 can be or include one or more cylindrical rods as illustrated in one or more of the figures; however, this need not be the case and other shapes can be utilized. For example, the reservoir body 112 can have any cross-sectional shape according to an implementation of the disclosure, which can include being tubular.

A vaporizer or cartridge 150 according to the disclosure can include a support structure for supporting one or more other components of the device or system, such as one or more reservoir bodies 112, a plurality of heating modules 106 and/or a plurality of heating elements. For instance, in at least one embodiment, the device or system can include one or more support tubes 118 for supporting a plurality of heating modules 106 relative to one another, such as in a spaced relationship along the length of a reservoir body 112 as described elsewhere herein. In at least one embodiment, one or more support tubes (or spacers) 118 can be disposed longitudinally between adjacent ones of the plurality of heating modules 106. In at least one embodiment, one or more support tubes 118 can be disposed radially between the exterior surface of a reservoir body 112 and the interior surfaces of the heating modules 106. In at least one embodiment, one or more support tubes 118 can be disposed radially about the exterior surfaces of the heating modules 106. One or more support tubes 118 can be made from any material(s) according to an implementation of the disclosure and in at least some exemplary embodiments can be made from materials such as glass, crystal or ceramic. As other examples, one or more support tubes 118 can be made of metal or, for instance, high temperature polymer or plastic. Further, in at least one embodiment, such a support tube 118 can have one or more openings in or through the exterior wall thereof, which can include being mesh or webbed.

In at least one embodiment, a vaporizer or cartridge 150 according to the disclosure can include one or more bases or caps 152, such as end caps, configured to couple to at least one end of the reservoir body 112 for holding the reservoir body 112 in place relative to one or more of the plurality of heating modules 106. In such an embodiment, which is but one of many, one or more end caps 152 can be configured to couple to one or more ends of a support tube 118 and/or heating module 106 and can include a centrally or otherwise located coupler for coupling with an end of the reservoir body 112 to hold the reservoir body 112 radially or otherwise within or relative to the support tube(s) 118 and/or heating module(s) 106.

In at least one embodiment, one or more of a plurality of heating modules 106 can be independently controllable (in whole or in part), which can allow a user to have optimal control over vaporization during vaporizer operations. For example, one or more controllers 160 can be operably coupled to the plurality of heating modules 106 (or heating elements) and configured for selective activation of any one or more of the heating modules 106 at any given time. For instance, a controller 160 can have one or more buttons, or other user inputs, 125 and be configured for allowing a user to selectively activate one heating module 106, all heating modules 106, or another number of heating modules 106 during a given vaporizer operation for controlling the amount of heat applied to the reservoir body 112 or vaporizable substance during such operation. In this manner, the devices and systems of the present disclosure can advantageously allow a user to more precisely control the volume of vapor generated during any given vaporizer operation (or “hit”) and thus the strength or potency of the substance delivered by the hit.

In at least one embodiment, a vaporizer or cartridge 150 according to the disclosure can be configured for rapidly heating all or a portion of a reservoir body 112 and then throttling back the heat for optimizing control of a vaporizer operation. This can include initially activating all heating modules 106 and then deactivating one or more heating modules 106 or, as another example, can include initially heating one or more heating modules 106 to a relatively high temperature and then reducing the temperature of such heat module(s) 106 (or both). In at least one embodiment, a controller 160 can be operably coupled to two or more heating modules 106 and configured to selectively or otherwise heat a reservoir body 112 to a first temperature for a first time period, reduce an amount of heat applied to the reservoir body 112, and maintain the reservoir body 112 at a second temperature for a second time period. In at least one embodiment, a controller 160 can be configured to simultaneously operate two or more heating modules 106 at a first heating level, simultaneously operate the heating modules 106 at a second heating level less than the first heating level, and thereafter operate one or both of the heating modules 106 at another heating level, which can include a higher heating level, a lower heating level, or both.

In at least one embodiment, a vaporizer or cartridge 150 according to the disclosure can include one or more other components for supporting vaporizer operations, which can include components for helping control heat transfer into, out of and/or within one or more system components. For instance, a vaporizer or cartridge 150 according to the disclosure can include one or more reflectors 170 for reflecting heat in one or more directions. In at least one embodiment, one or more reflectors 170 can be disposed about or otherwise in thermal communication with one or more heating modules 106 for reflecting outwardly directed heat inwardly toward the reservoir body 112.

As another example, a vaporizer or cartridge according to the disclosure can include one or more fins, or other heat sinks, 180 for drawing heat away from one or more other components of the system and dissipating or distributing such heat elsewhere. For instance, a cool zone can include one or more fins disposed longitudinally between adjacent hot zones for controlling heat distribution along a reservoir body 112, between or among adjacent heating modules or otherwise in accordance with an implementation of the disclosure. As another example, one or more heat sinks 180 can be thermally coupled to one or both ends of a reservoir body 112, support tube 118, or both. In at least one embodiment, such a heat sink, if present, can be part of or can constitute an end cap 152 as described above or can otherwise be configured for supporting a reservoir body 112 and/or other components in their operative positions.

While embodiments of the disclosure have been described as including one or more radiant heating modules, this need not be the case and one or more other types of heating devices or heaters can be utilized if required or desired according to an implementation of the disclosure, whether separately or in combination with one another and/or with one or more radiant heating modules. For instance, in at least one embodiment, a device or system according to the disclosure can include one or more of an infrared radiant heating module, a convection heating module, a forced convection heating module and a laser heating module. Further, as will be appreciated by a person of ordinary skill in the art having the benefits of the present disclosure, the reservoir bodies, heating modules and other components disclosed herein can be, and typically will be, incorporated into an overall vaporizer structure or cartridge structure having, for instance, a housing, an air inlet, an air outlet (e.g., in a mouthpiece) and a vaporization chamber, and which either includes or is configured to couple with a power source, such as a conventional 510 threaded power source or another power source. For instance, in at least one embodiment, one or more reservoir bodies and a plurality of heating modules according to the disclosure can be disposed partially or wholly within the vaporization chamber of a vaporizer housing (including, but not limited to, in the form of a modular insert) and disposed in operative communication with other vaporizer components such as mouthpieces, air flow paths and power sources. Examples of such components are disclosed in Applicant's co-pending U.S. patent application Ser. No. 16/380,948 filed Apr. 10, 2019 and U.S. Pat. No. 16,402,139 filed May 2, 2019, the entire contents of which are hereby incorporated by reference herein.

In at least one embodiment, a device for storing a vaporizable substance, such as a reservoir body according to the disclosure, can be or include a porous body having longitudinal ends and a radially exterior surface. The body can be configured to absorb a volume of liquid and to retain the volume of liquid until at least a portion of the volume of liquid is vaporized, and the body can be configured to retain its shape and pore volume independently of contact with water. The body can be configured to hold oil, medication or another substance, such as in fluid form, and, when heated to an applicable temperature, which can include a temperature sufficient to vaporize at least a portion of such substance without causing carbonization, can release at least a portion of such substance in the form of gas and/or vapor. In at least one embodiment, the device can have an internal volume of pores for holding a prescribed amount (e.g., by weight, mass, volume or another measure) of fluid and can be configured to vary in one dimension (e.g., thickness or diameter) to accommodate one or more amounts of fluid without varying in one or more other dimensions or shapes. At least a portion of the body of the device can be solid or can comprise a solid. In at least one embodiment, at least one of the body's shape, pore size, pore volume and a combination thereof can be fixed.

In at least one embodiment, a liquid stored within the device can have a vaporization temperature and the body or device can have a melting or burning temperature, and the melting or burning temperature can be greater than the vaporization temperature. A pore size of the body can be varied or otherwise adapted for fluids of different viscosities, which can be or include any viscosity according to an implementation of the disclosure. For example, a pore size of the device or a body of the device can be or include a pore size(s) configured for intaking and/or holding by capillary action fluid(s) having a viscosity from about 5 Pa-sec to about 100 Pa-sec at 68 degrees F. or from about 0.5 Pa-sec to about 10 Pa-sec at 113 degrees F. The pore size can be varied to handle different viscosities of oil or other fluid. For example, one or more pore sizes described herein can be used for oils falling within the above-mentioned viscosity ranges. In at least one embodiment, the body can include a matrix of pores, such as interconnected pores, and the matrix can be configured for holding one or more fluids in place within the body, such as via capillary action, which can include holding fluid(s) in place within the body while at least partially minimizing or eliminating the existence of fluid on an exterior surface of the body, in whole or in part. In at least one embodiment, the device can be configured to hold fluid(s) within at least a portion of the pores or matrix until any holding force(s) are overcome via the application of thermal heating, such as heating to a temperature sufficient to overcome the capillary action, which can include turning at least a portion of liquid or other fluid in the matrix to gas or vapor.

In at least one embodiment, a cartridge for a vaporizer can include a reservoir for holding a vaporizable substance. In at least one embodiment, the reservoir can comprise an elongated reservoir body having a longitudinal axis, a first end and a second end longitudinally opposite the first end. In at least one embodiment, a cartridge for a vaporizer can include a first heating module disposed at a first location along the longitudinal axis of the reservoir body. In at least one embodiment, a cartridge for a vaporizer can include a second heating module disposed at a second location along the longitudinal axis of the reservoir body. In at least one embodiment, a cartridge for a vaporizer can include a first support tube disposed at a third location along the longitudinal axis of the reservoir body between the first and second heating modules. In at least one embodiment, a cartridge for a vaporizer can include a third heating module disposed at a fourth location along the longitudinal axis of the reservoir body. In at least one embodiment, a cartridge for a vaporizer can include a second support tube disposed at a fifth location along the longitudinal axis of the reservoir body between the second and third heating modules. In at least one embodiment, a cartridge for a vaporizer can include a controller operably coupled to the heating modules. In at least one embodiment, the controller can be configured for selective and/or independent activation of the heating modules. In at least one embodiment, the controller can be configured to selectively heat different portions of the reservoir.

In at least one embodiment, a reservoir body or device can be or include a porous body having a plurality of pores that have a pore size from 0.0001 micron to 100 microns, or less, or more. As another example, a device can include a porous body having a plurality of pores that have a pore size of from 0.1 micron to 20 microns. As another example, a device can include a porous body having a plurality of pores that have a pore size of from 0.4 micron to 5 microns. As another example, a device can include a porous body having a plurality of pores that have a pore size of from 0.5 micron to 5 microns. As other examples, a device can include a porous body having a plurality of pores that have a pore size of from 0.5 micron to 3 microns, or of up to 50 microns, such as 15 microns. As another example, a device can include a porous body having a plurality of pores that have one or more of the above-mentioned pore sizes and a particle retention of 0.5 micron, or of from 0.5 micron to 50 microns or so. Such pore sizes can be average or nominal pore sizes or, for instance, maximum pore sizes. In at least one embodiment, at least a portion of the body can be composed of at least one of ceramically bonded alumina, thermally fused alumina, fused silicon carbide, fused metallic beads, fused mineral beads, and a combination thereof. In at least one embodiment, at least a portion of the body can be composed of alumina (Al₂O₃) and silicone dioxide (SiO₂), separately or in combination with one or more other materials. For example, in at least one embodiment, a porous body can include 75-95% alumina and 5-25% silicone dioxide, or 80-90% alumina and 10-20% silicone dioxide, or 85% alumina and 10-15% silicone dioxide, or about 85% alumina and about 10-15% silicone dioxide. In at least one embodiment, a body can include one or more metals or other materials, such as one or more non-toxic metals or other materials (e.g., TiO2, FE2O3, Na2O, B203, CaO, MgO). In at least one embodiment, a body can include a nominal particle retention of 0.001-1 micron, such as 0.5 micron or about 0.5 micron, a maximum pore diameter of 15 microns or about 15 microns, a porosity of 20-70%, such as 40-50%, a bulk density of 0.001-4 g/cc, such as 2 g/cc or about 2 g/cc, and a modulus of rupture (MOR) of 2000-5000 psi, such as 4000 psi or about 4000 psi. In at least one embodiment, at least a portion of the body can be composed of naturally occurring materials, such as materials that do not need to be fused at relatively high temperatures, for example coral, diatomaceous earth, or porous lava rock, separately or in combination, in whole or in part.

Other and further embodiments utilizing one or more aspects of the devices, systems and methods described herein can be devised without departing from the spirit of Applicant's disclosure. For example, the devices, systems and methods disclosed herein can be used alone or to form one or more parts of other vaporizers or vaporizing systems. Further, the various methods and embodiments hereof can be included in combination with each other to produce variations of the disclosed methods and embodiments. Discussion of singular elements can include plural elements and vice-versa. References to at least one item followed by a reference to the item can include one or more items. Also, various aspects of the embodiments can be used in conjunction with each other to accomplish the goals of the disclosure.

Unless the context requires otherwise, the words “comprise,” “include,” and “has” (including variations and conjugations thereof, such as “comprises,” “including,” “have” and so forth) should be understood to imply the inclusion of at least the stated element or step or group of elements or steps or equivalents thereof, and not the exclusion of a greater numerical quantity or any other element or step or group of elements or steps or equivalents thereof. The devices, apparatuses and systems can be used in a number of directions and orientations. The order of steps can occur in a variety of sequences unless otherwise specifically limited. The various steps described herein can be combined with other steps, interlineated with the stated steps, and/or split into multiple steps. Similarly, elements have been described functionally and can be embodied as separate components and/or can be combined into components having multiple functions.

The embodiments have been described in the context of preferred and other embodiments and not every embodiment of Applicant's disclosure has been described. Obvious modifications and alterations to the described embodiments are available to those of ordinary skill in the art having the benefits of the present disclosure. The disclosed and undisclosed embodiments are not intended to limit or restrict the scope or applicability of Applicant's disclosure, but rather, in conformity with the patent laws, Applicant intends to fully protect all such modifications and improvements that come within the scope or range of equivalents of the claims.

What is claimed is:

1. A cartridge for a vaporizer, comprising:
a reservoir for holding a vaporizable substance, wherein the reservoir comprises an elongated reservoir body having a longitudinal axis, a first end and a second end longitudinally opposite the first end;
a plurality of heating modules comprising a first heating module and a second heating module;
wherein the first heating module is configured to heat a first portion of the reservoir body and the second heating module is configured to heat a second portion of the reservoir body;
wherein the first heating module is disposed at a first location along the longitudinal axis of the reservoir body;
wherein the second heating module is disposed at a second location along the longitudinal axis of the reservoir body; and
wherein the second heating module is longitudinally spaced from the first heating module.

2. The cartridge of claim 1, wherein the plurality of heating modules further comprises one or more additional heating modules disposed at one or more respective additional locations along the longitudinal axis of the reservoir body and configured to heat one or more respective additional portions of the reservoir body, and wherein the one or more additional heating modules are longitudinally spaced from the first heating module and the second heating module.

3. The cartridge of claim 2, wherein the spacing between adjacent ones of the plurality of heating modules is uniform.

4. The cartridge of claim 2, wherein the spacing between the first and second heating modules is different from the spacing between the second heating module and at least one of the one or more additional heating modules.

5. The cartridge of claim 1, wherein at least one of the plurality of heating modules comprises a radiant heat source disposed in thermal communication with the reservoir body.

6. The cartridge of claim 5, wherein the radiant heat source is separated from the reservoir body by an air gap.

7. The cartridge of claim 5, wherein the radiant heat source is configured to heat the reservoir body from at least two directions.

8. The cartridge of claim 1, wherein each of the plurality of heating modules comprises an annular heating body having an opening therethrough and wherein the reservoir body is disposed through the openings of the annular heating bodies.

9. The cartridge of claim 8, wherein the reservoir body is suspended within the openings of the annular heating bodies with an air gap between the exterior surface of the reservoir body and the interior surfaces of the annular heating bodies.

10. The cartridge of claim 9, wherein the reservoir body is cylindrical and is suspended from its ends.

11. The cartridge of claim 1, further comprising a support tube between the first and second heating modules.

12. The cartridge of claim 1, wherein the first and second heating modules are independently controllable.

13. The cartridge of claim 1, further comprising a controller operably coupled to the first and second heating modules and configured for selective activation of one or both of the first and second heating modules.

14. The cartridge of claim 1, further comprising one or more reflectors configured to reflect heat from one or more of the plurality of heating modules.

15. The cartridge of claim 1, further comprising one or more heat sinks in thermal communication with one or more of the plurality of heating modules.

16. The cartridge of claim 1, wherein the reservoir body includes a porous body configured to store a vaporizable substance in a plurality of internal pores.

17. The cartridge of claim 1, further comprising one or more end caps configured to couple to at least one end of the reservoir body for holding the reservoir body in place relative to one or more of the plurality of heating modules.

18. The cartridge of claim 1, further comprising a controller operably coupled to the first and second heating modules, wherein the controller is configured to selectively—
heat the reservoir body to a first temperature for a first time period;
reduce an amount of heat applied to the reservoir body; and
maintain the reservoir body at a second temperature for a second time period.

19. The cartridge of claim 18, wherein the controller is configured to to simultaneously operate both of the first and second heating modules at a first heating level, simultaneously operate both of the first and second heating modules at a second heating level less than the first heating level, and thereafter selectively operate one or both of the first and second heating modules at a third heating level.

20. A cartridge for a vaporizer, comprising:
a reservoir for holding a vaporizable substance, wherein the reservoir comprises an elongated reservoir body having a longitudinal axis, a first end and a second end longitudinally opposite the first end;
a first heating module disposed at a first location along the longitudinal axis of the reservoir body;
a second heating module disposed at a second location along the longitudinal axis of the reservoir body;
a first support tube disposed at a third location along the longitudinal axis of the reservoir body between the first and second heating modules;
a third heating module disposed at a fourth location along the longitudinal axis of the reservoir body;
a second support tube disposed at a fifth location along the longitudinal axis of the reservoir body between the second and third heating modules; and
a controller operably coupled to the heating modules and configured for selective and independent activation of the heating modules, such that the controller is configured to selectively heat different portions of the reservoir.
